(19) United States
(12) Patent Application Publication
(10) Pub. No.: US 2018/0296586 A1
(43) Pub. Date: Oct. 18, 2018

(54) USE OF NADPH FOR PREPARATION OF A DRUG FOR ANTIPLATELET AGGREGATION

(71) Applicant: Chongqing Institute of Bench-to-Bed Bioengineering Technology Co., Ltd., Chongqing (CN)

(72) Inventors: ZHENG-HONG QIN, Suzhou (CN); Mei LI, Suzhou (CN)

(73) Assignee: Chongqing Institute of Bench-to-Bed Bioengineering Technology Co., Ltd., Chongqing (CN)

(21) Appl. No.: 15/769,063

(22) PCT Filed: Jan. 22, 2016
(86) PCT No.: PCT/CN2016/071727

§ 371 Date: Apr. 17, 2018

(30) Foreign Application Priority Data
Oct. 22, 2015 (CN) 201510689544.8

(51) Int. Cl. A61K 31/7084; A61K 9/20; A61K 9/48; A61P 7/02

(52) U.S. Cl. CPC A61K 31/7084; A61K 9/2077; A61K 9/0031; A61P 7/02; A61K 9/4808

(57) ABSTRACT

Use of NADPH for preparation of a drug for antiplatelet aggregation, according to the research, it is found that in-vitro administration of exogenous NADPH inhibits ADP-induced platelet aggregation of rats in a dose-dependent mode; in-vitro administration of exogenous NADPH inhibits Thrombin-induced platelet aggregation of rats in a dose-dependent mode; preventive administration of NADPH in the rats can remarkably inhibit ADP-induced platelet aggregation; preventive administration of NADPH in the rats can remarkably inhibit Thrombin-induced platelet aggregation. Therefore, NADPH has a platelet aggregation inhibition function and can be used as a potential drug for antiplatelet aggregation.

### TECHNICAL FIELD

The present invention belongs to the field of pharmaceuticals and particularly relates to use of NADPH (triphosphopyridine nucleotide) for preparation of a drug for antiplatelet aggregation.

### BACKGROUND

Platelet aggregation is a main step of thrombosis. In normal circulating blood, platelets are in a resting state. When blood vessel walls suffer from injury such as artery atherosclerotic plaque cracking and subendothelial matrix of blood vessels is exposed, the platelets will be combined with a vorl Willebrand factor (VWF) through surface membrane glycoprotein (GP) Ib of the platelets so as to be adhered to subendothelial collagenous tissue, and meanwhile, the platelets are directly combined with collagen through collagen receptors GP Ia-IIa and GPVI of surfaces of the platelets so as to be firmly adhered to the subendothelial collagenous tissue. Adhered platelets or platelets acted by platelet activators (e.g. collagen, thrombin, etc.) will be subjected to a series of reactions, including arachidonic acid metabolism, thromboxane A2 (TXA2) production and intracellular particulate content adenosine diphosphate (ADP) release, finally, the configuration of complexes GP IIb-IIIa of the platelets is changed to form adhered molecular receptors, the platelets are adhered to one another and are aggregated through the combination with fibrinogen, and then, early-stage hemostatic thrombosis is formed at broken positions of blood vessels. In addition, release products of the platelets can be used for further causing vasoconstriction, stimulating leucocytes, injuring endothelial cells and promoting blood coagulation, thereby being beneficial to thrombosis.

Drugs for antiplatelet aggregation can be used for preventing or reversing platelet aggregation and are mostly extensively applied to arterial thrombosis inhibition, particularly surgeries, vascular stent post-implantation, coronary artery bypass grafting, myocardial infarction and myocardial ischemia. In accordance with a mechanism for platelet aggregation, at present, the drugs for antiplatelet aggregation mainly comprise: (1) drugs for inhibiting thromboxane A2 (TXA2) induced platelet aggregation and aspirin is taken as a representative drug; (2) drugs for inhibiting adenosine diphosphate (ADP) induced platelet aggregation and ticlopidine and clopidogrel are taken as representative drugs; and (3) platelet glycoprotein IIb/IIIa receptor antagonists for inhibiting a final common way of platelet aggregation and abciximab, tirofiban and eptifibatide are taken as representative drugs. In addition, platelet activation factor (PAF) receptor antagonists, thrombin and blood coagulation factor Xa (FXa) inhibitors, calcium ion (Ca²⁻) channel antagonists and 5-HT2 receptor antagonists all play a role in inhibiting platelet aggregation.

Reduced triphosphopyridine nucleotide (NADPH) is produced from glucose through metabolism in a phosphopentose pathway (PPP) and can be used for providing hydrogen ions for reductive biosynthesis as the most important electron donor and biosynthesis reducer in cells. NADPH is a coenzyme of glutathione (GSH) reductase and can be used for enabling oxidated glutathione (GSSG) to produce reduced GSH and maintaining the normal content of the reduced GSH. The GSH is an intracellular important antioxidant, can be used for protecting some sulfhydryl-containing proteins, sulfhydryl-containing fats and sulfhydryl-containing proteases from being destroyed by oxidants and particularly plays an influential role in maintaining the integrity of erythrocyte membranes. The NADPH also participates in in-vivo hydroxylation and the bioconversion of drugs, toxic substances and some hormones, besides participation in the biosynthesis of cholesterol, fatty acids, monooxygenase systems, steroid hormones, etc. For example, the NADPH can utilize electron donors of detoxification cells to reduce oxidated compounds of biosome through in-vivo metabolism so as to maintain the redox balance, thus the NADPH plays an influential role in an oxidation defense system. The NADPH can also enter a respiratory chain to generate ATP by virtue of a shuttle action of isocitric acid: due to very low permeability of a mitochondrial inner membrane to substances, the NADPH produced outside mitochondria cannot directly enter the respiratory chain to be oxidated. H on the NADPH can be delivered to NAD+ under the action of isocitrate dehydrogenase and then enters the respiratory chain to produce energy through the NAD+. The maintaining of cell energy metabolism and the reducing of ROS (Reactive Oxygen Species) are very important for the survival of cells, particularly ischemic and anoxic tissue; it is generally believed that metabolic disorder of energy and oxidative stress are important mechanisms of ischemic cardiovascular and cerebral vascular diseases; and researches show that the improvement of energy metabolic capacity of cells and the reducing of ROS production of the cells can alleviate cell injury caused by ischemia and anoxia.

At present, it is not reported that the NADPH is used for treating platelet aggregation yet.

### SUMMARY

Therefore, the present invention provides use of NADPH for preparation of a drug for antiplatelet aggregation.

In order to solve the above-mentioned technical problem, the present invention is implemented through the technical scheme as follows:

The present invention provides use of NADPH for preparation of a drug for antiplatelet aggregation.

Preferably, according to the use provided by the present invention, the drug comprises a pharmaceutically effective amount of NADPH and a pharmaceutically acceptable carrier.

Further preferably, according to the use provided by the present invention, the carrier is selected from a group consisting of frequently-used medicinal adjuvants, physiological saline and distilled water.

Further preferably, according to the use provided by the present invention, the drug is in form of clinically-acceptable tablets, capsules, powder, mixtures, pills, granules, sugar syrup, paste, suppositories, aerosol, ointment or injections which are prepared through adding a conventional adjuvant into the NADPH according to a conventional process.

The pharmaceutically acceptable adjuvants comprise fillers, disintegrants, lubricants, suspending agents, binders, sweetening agents, flavoring agents, preservatives, matrixes, etc. The fillers comprise starch, pregelatinized starch, lactose, mannitol, chitin, microcrystalline cellulose, saccharose, etc.; the disintegrants comprise starch, pregelatinized starch, microcrystalline cellulose, carboxymethyl starch sodium, crosslinked polyvinylpyrrolidone, low-substituted hydroxypropyl cellulose, crosslinked sodium carboxymethyl cellulose, etc.; the lubricants comprise magnesium stearate, sodium laurylsulfate, talcum powder, silicon dioxide, etc.; the suspending agents comprise polyvinylpyrrolidone, microcrystalline cellulose, saccharose, agar, hydroxypropyl methylcellulose, etc.; the binders comprise starch slurry, polyvinylpyrrolidone, hydroxypropyl methylcellulose, etc.; the sweetening agents comprise sodium saccharin, aspartame, saccharose, sodium cyclamate, glycyrrhetinic acid, etc.; the flavoring agents comprise sweetening agents and various essences; the preservatives comprise nipagin, benzoic acid, sodium benzoate, sorbic acid and salts thereof, benzalkonium bromide, chlorhexidine acetate, eucalyptus oil, etc.; and the matrixes comprise PEG6000, PEG4000, insect wax, etc.

Further preferably, according to the use provided by the present invention, administration of the drug comprises at least one selected from a group consisting of oral administration, injection administration, sublingual administration, rectal administration, transdermal administration and nebulization inhalation.

The technical scheme of the present invention has the following advantages:

According to the present invention, the research shows that in-vitro administration of exogenous NADPH inhibits ADP-induced platelet aggregation of rats in a dose-dependent mode; in-vitro administration of exogenous NADPH inhibits Thrombin-induced platelet aggregation of rats in the dose-dependent mode; preventive administration of the NADPH in the rats can remarkably inhibit the ADP-induced platelet aggregation; and preventive administration of the NADPH in the rats can remarkably inhibit the Thrombin-induced platelet aggregation. Therefore, NADPH has a platelet aggregation inhibition function and can be used as a potential drug for antiplatelet aggregation.

### BRIEF DESCRIPTION OF THE DRAWINGS

In order to describe specific embodiments of the present invention or the technical scheme in the prior art more clearly, drawings required to be used in descriptions of the specific embodiments or the prior art will be introduced simply below. Apparently, the drawings described below are some embodiments of the present invention, and other drawings can be obtained by those having ordinary skill in the art according to these drawings on the premise of not making inventive labor.

FIG. 1 represents influence on adenosine diphosphate (ADP)-induced platelet aggregation of rats caused by in-vitro administration of exogenous NADPH in an experimental example 1 of the present invention, wherein * represents P<0.05, and *** represents P<0.01;

FIG. 2 represents influence on thrombin-induced platelet aggregation of rats caused by in-vitro administration of exogenous NADPH in an experimental example 2 of the present invention, wherein * represents P<0.05, and *** represents P<0.001;

FIG. 3 represents influence on ADP-induced platelet aggregation caused by preventive administration of NADPH in rats in an experimental example 3 of the present invention, wherein ** represents P<0.01;

FIG. 4 represents influence on Thrombin-induced platelet aggregation caused by preventive administration of NADPH in rats in an experimental example 4 of the present invention, wherein ** represents P<0.01.

### DETAILED DESCRIPTION

The technical scheme of the present invention will be described below clearly and completely with reference to the drawings. Apparently, embodiments described are part of embodiments of the present invention, rather than all embodiments. All other embodiments obtained by those having ordinary skill in the art on the premise of not making inventive labor on the basis of the embodiments of the present invention fall within the protection scope of the present invention.

### Embodiment 1: Preparation of NADPH Capsules

In the embodiment, the NADPH capsules are prepared from:

20 g of NADPH, 60 g of suspending agent microcrystalline cellulose, 0.04 g of preservative tertiary butyl-4-hydroxyanisole, 2 g of lubricant magnesium stearate and 200 g of filler lactose.

A preparation method of the NADPH capsules comprises the following steps:

Separately weighing the NADPH and various pharmaceutical adjuvants in prescription amount, uniformly mixing the weighed NADPH and the weighed pharmaceutical adjuvants, sieving the mixture for 3 times by a sieve of 60-mesh, and loading the sieved mixture into capsule shells, thereby obtaining the NADPH capsules.

### EXPERIMENTAL EXAMPLE

### Experimental Example 1

### Influence on Adenosine Diphosphate (ADP)-Induced Platelet Aggregation of Rats Caused by In-Vitro Administration of Exogenous NADPH

(1) Experiment Materials

Adenosine diphosphate (ADP) and thrombin reagents purchased from Sangon company, and the NADPH reagent purchased from sigma company; an exogenous NADPH drug capable of being obtained through artificial synthesis, semi-synthesis and biological extraction; and 20 clean-grade adult male SD rats with the body weight of 270 g to 350 g, provided by Experimental Animal Center of Department of Medicine of Suzhou university [Permission Number: (Su)SYXK 2007-0035].

Room temperature 22 DEG C., humidity 50% to 60%, well ventilation, artificial day and night (12 h/12 h), and free food and water intake.

Before experiment, the male rats are accommodated for 2 d in a breeding environment.

(2) Experiment Scheme

20 rats are subjected to blood drawing from abdominal aorta, 3.8% sodium citrate is used for carrying out anticoagulation treatment according to a drawn blood volume/anticoagulant ratio of 9:1, centrifugation is carried out for 5 min at a rate of 1000 r/min, upper-layer platelet-rich plasma (PRP) is sucked out, remaining blood is subjected to centrifugation for 10 min at a rate of 3000 r/min to prepare platelet-poor plasma (PPP) until the number of platelets is 2*10<8>/mL, and platelet aggregation is detected by adopting a platelet aggregometer Chronolog-590.

(3) Experiment Method

1) Detection of Platelet Aggregation

The platelet aggregometer is turned on and preheated for 30 min, and light transmittance is adjusted to 100% with PPP as blank control. 0.25mL of NADPH samples of different concentrations (30 μM, 60 μM and 90 μM) are added into PRP, preheating is conducted for 5 min at the temperature of 37 DEG C., the preheated material is put into the aggregometer, then, a certain amount of ADP (20 μM) is added to induce platelet aggregation, graphic changes in 4 min are recorded, and conditions of ADP-induced platelet aggregation caused by NADPH are observed.

2) Data Statistics and Analysis

Data are all represented by mean+/−standard error of mean (Mean+/−SEM), statistical analysis adopts one-way analysis of variance (one-way ANOVA), and P<0.05 shows that the difference is statistically significant.

(4) Experiment Result

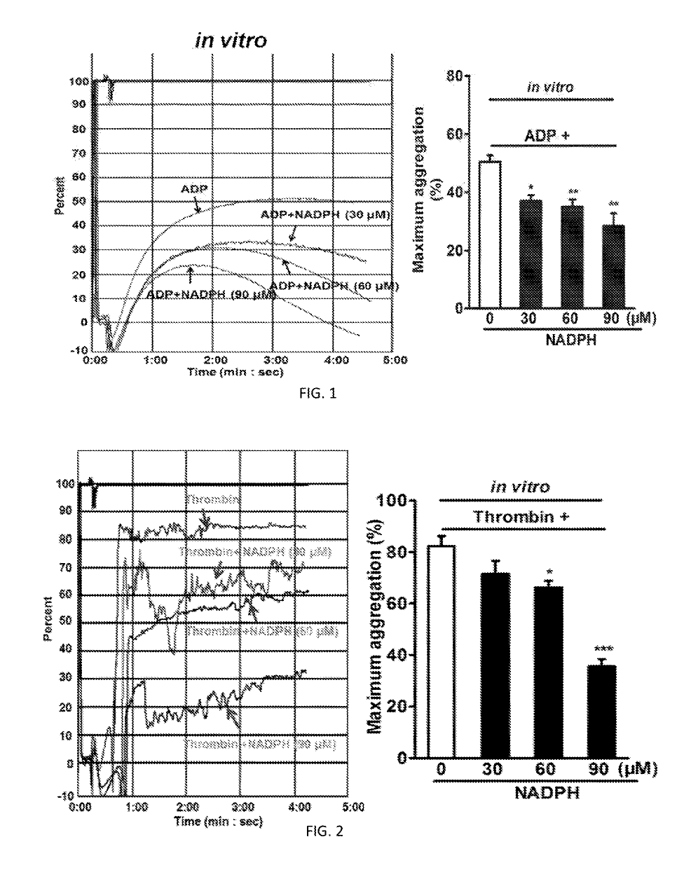

Referring to FIG. 1, compared with an ADP group, P is smaller than 0.05, and P is smaller than 0.01; and in-vitro administration of exogenous NADPH inhibits ADP-induced platelet aggregation of rats in a dose-dependent mode.

### Experimental Example 2

### Influence on Thrombin-Induced Platelet Aggregation of Rats Caused by In-Vitro Administration of Exogenous NADPH

(1) Experiment Materials are the Same as Those in the Experimental Example 1.

(2) An Experiment Scheme is the Same as that in the Experimental Example 1.

(3) Experiment Method

1) Detection of Platelet Aggregation

The platelet aggregometer is turned on and preheated for 30 min, and light transmittance is adjusted to 100 % with PPP as blank control. 0.25 mL of NADPH samples of different concentrations (30 μM, 60 μM and 90 μM) are added into PRP, preheating is conducted for 5 min at the temperature of 37 DEG C., the preheated material is put into the aggregometer, then, a certain amount of thrombin (2U) is added to induce platelet aggregation, graphic changes in 4 min are recorded, and conditions of thrombin-induced platelet aggregation caused by NADPH are observed.

2) Data Statistics and Analysis

Data are all represented by mean+/−standard error of mean (Mean+/−SEM), statistical analysis adopts one-way analysis of variance (one-way ANOVA), and P<0.05 shows that the difference is statistically significant.

(4) Experiment Result

Referring to FIG. 2, compared with a thrombin group, P is smaller than 0.05, and P is smaller than 0.01; and thrombin can be used for inducing platelet aggregation of rats, and in-vitro administration of exogenous NADPH inhibits thrombin-induced platelet aggregation of the rats in a dose-dependent mode.

### Experimental Example 3

### Influence on ADP-Induced Platelet Aggregation of Rats Caused by Preventive Administration of NADPH in Rats

(1) Experiment Materials are the Same as Those in the Experimental Example 1.

(2) Experiment Scheme

Normal SD male rats are randomly divided into 2 groups, i.e., a physiological saline group (saline group) and an NADPH (7.5 mg/kg) dosage group in a manner that each group comprises 10 rats. NADPH is injected into the rats through caudal veins 30 min before blood drawing. The rats are subjected to blood drawing from abdominal aorta, 3.8% sodium citrate is used for carrying out anticoagulation treatment according to a drawn blood volume/anticoagulant ratio of 9:1, centrifugation is carried out for 5 min at a rate of 1000 r/min, upper-layer platelet-rich plasma (PRP) is sucked out, remaining blood is subjected to centrifugation for 10 min at a rate of 3000 r/min to prepare platelet-poor plasma (PPP) until the number of platelets is 2*10<8>/mL, and platelet aggregation is detected by adopting a platelet aggregometer Chronolog-590.

(3) Experiment Method

1) Detection of Platelet Aggregation

The platelet aggregometer is turned on and preheated for 30 min, and light transmittance is adjusted to 100% with PPP as blank control. APRP sample of the physiological saline group (saline group) and a PRP sample of the NADPH (7.5 mg/kg) dosage group are preheated for 5 min at the temperature of 37 DEG C., the preheated PRP samples are put into the aggregometer, then, a certain amount of ADP (20 μM) is added to induce platelet aggregation, graphic changes in 4 min are recorded, and conditions of ADP-induced platelet aggregation caused by NADPH are observed.

2) Data Statistics and Analysis

Data are all represented by mean+/−standard error of mean (Mean+/−SEM), statistical analysis adopts t-test analysis, and P<0.05 shows that the difference is statistically significant.

(4) Experiment Result

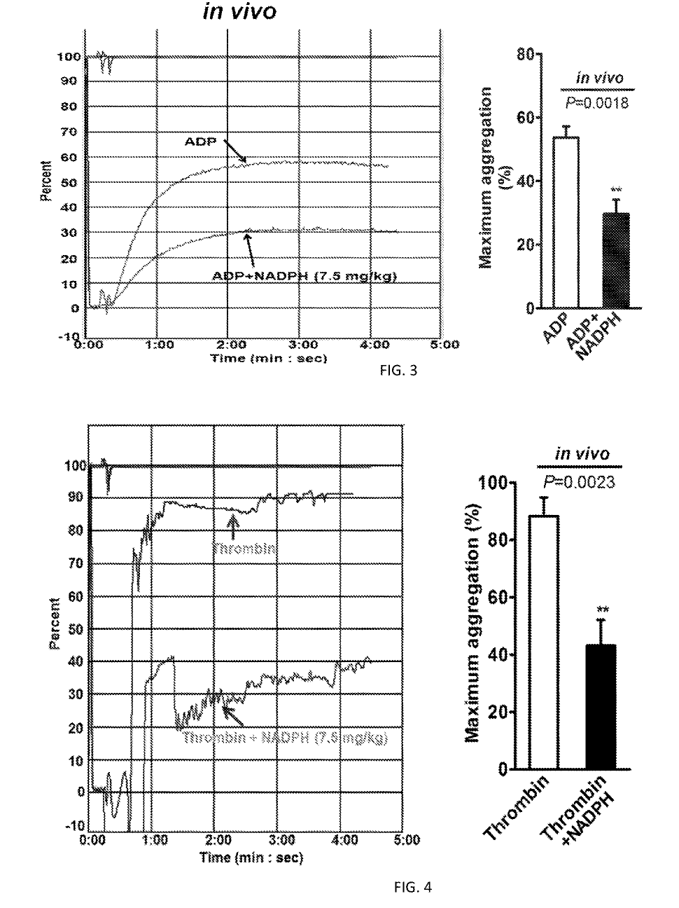

Referring to FIG. 3, compared with an ADP group, P is smaller than 0.01; and preventive administration of NADPH in rats can remarkably inhibit ADP-induced platelet aggregation.

### Experimental Example 4

### Influence on Thrombin-Induced Platelet Aggregation of Rats Caused by Preventive Administration of NADPH in Rats

(1) Experiment Materials are the Same as Those in the Experimental Example 1.

(2) An Experiment Scheme is the Same as that in the Experimental Example 3.

(3) Experiment Method

1) Detection of Platelet Aggregation

The platelet aggregometer is turned on and preheated for 30 min, and light transmittance is adjusted to 100% with PPP as blank control. A PRP sample of the physiological saline group (saline group) and a PRP sample of the NADPH (7.5 mg/kg) dosage group are preheated for 5 min at the temperature of 37 DEG C., the preheated PRP samples are put into the aggregometer, then, a certain amount of thrombin (2U) is added to induce platelet aggregation, graphic changes in 4 min are recorded, and conditions of thrombin-induced platelet aggregation caused by NADPH are observed.

2) Data Statistics and Analysis

Data are all represented by mean+/−standard error of mean (Mean+/−SEM), statistical analysis adopts t-test analysis, and P<0.05 shows that the difference is statistically significant.

(4) Experiment Result

Referring to FIG. 4, compared with a thrombin group, P is smaller than 0.01; and preventive administration of NADPH in rats can remarkably inhibit thrombin-induced platelet aggregation.

In summary, the research shows that the NADPH has a platelet aggregation inhibition function and can be used as a potential drug for antiplatelet aggregation.

Apparently, the above-mentioned embodiments are only illustrated for distinct description, but are not intended to limit embodiments. For those having ordinary skill in the art, changes or variations of other different forms can also be made on the basis of the above-mentioned description. Herein, all embodiments are not required to and cannot be exhaustive. Readily apparent changes or variations evolved therefrom still fall within the protection scope of the present invention.

1. A method for antiplatelet aggregation, comprising administration of a drug comprising NADPH.

2. The method according to claim 1, wherein the drug comprises a pharmaceutically effective amount of NADPH and a pharmaceutically acceptable carrier.

3. The method according to claim 2, wherein the carrier is selected from a group consisting of frequently-used medicinal adjuvants, physiological saline and distilled water.

4. The method according to claim 1, wherein the drug is in form of clinically-acceptable tablets, capsules, powder, mixtures, pills, granules, sugar syrup, paste, suppositories, aerosol, ointment or injections which are prepared through adding a conventional adjuvant into the NADPH according to a conventional process.

5. The method according to claim 1, wherein administration of the drug comprises at least one selected from a group consisting of oral administration, injection administration, sublingual administration, rectal administration, transdermal administration and nebulization inhalation.

6. The method according to claim 2, wherein the drug is in form of clinically-acceptable tablets, capsules, powder, mixtures, pills, granules, sugar syrup, paste, suppositories, aerosol, ointment or injections which are prepared through adding a conventional adjuvant into the NADPH according to a conventional process.

7. The method according to claim 3, wherein the drug is in form of clinically-acceptable tablets, capsules, powder, mixtures, pills, granules, sugar syrup, paste, suppositories, aerosol, ointment or injections which are prepared through adding a conventional adjuvant into the NADPH according to a conventional process.

8. The method according to claim 2, wherein administration of the drug comprises at least one selected from a group consisting of oral administration, injection administration, sublingual administration, rectal administration, transdermal administration and nebulization inhalation.

9. The method according to claim 3, wherein administration of the drug comprises at least one selected from a group consisting of oral administration, injection administration, sublingual administration, rectal administration, transdermal administration and nebulization inhalation.

10. The method according to claim 4, wherein administration of the drug comprises at least one selected from a group consisting of oral administration, injection administration, sublingual administration, rectal administration, transdermal administration and nebulization inhalation.

11. The method according to claim 6, wherein administration of the drug comprises at least one selected from a group consisting of oral administration, injection administration, sublingual administration, rectal administration, transdermal administration and nebulization inhalation.

12. The method according to claim 7, wherein administration of the drug comprises at least one selected from a group consisting of oral administration, injection administration, sublingual administration, rectal administration, transdermal administration and nebulization inhalation.
